(19) United States
(12) Patent Application Publication
(10) Pub. No.: US 2005/0113594 A1
(43) Pub. Date: May 26, 2005

(54) Process using a cyclic carbonate reactant

(76) Inventor: Jurgen Van Holen, Gent (BE)

Correspondence Address: WENDEROTH, LIND & PONACK, L.L.P., 2033 K STREET N. W., SUITE 800, WASHINGTON, DC, 20006-1021, US

(21) Appl. No.: 10/501,088

(22) PCT Filed: Jan. 30, 2003
(86) PCT No.: PCT/EP03/00942

(30) Foreign Application Priority Data
Feb. 7, 2002 (EP) 02002802.3

(51) Int. Cl. C07C 69/96

(52) U.S. Cl. 558/276

(57) ABSTRACT

The present invention relates to a process wherein a cyclic carbonate compound is reacted with a compound containing a nucleophilic group, especially an amine group, in the presence of a catalyst comprising a lithium compound. Such reactions permit to obtain urethane groups useful in polymer preparation, such as polyurethanes, or other urethane-containing polymers.

The present invention relates to a process wherein a cyclic carbonate compound is reacted with a compound containing a nucleophilic function.

The reaction of a cyclic carbonate compound with a compound containing a nucleophilic group, especially an amine compound, is known. First publications on that subject date from late fifties early sixties: see for example U.S. Pat. No. 2,802,022, U.S. Pat. No. 2,935,494 and U.S. Pat. No. 3,072,613.

When an amine compound (1) is reacted with a cyclic carbonate compound containing a 5-membered ring (1,3-dioxolan-2-one ring) (2), the product obtained is a betahydroxyurethane (3 and/or 4):

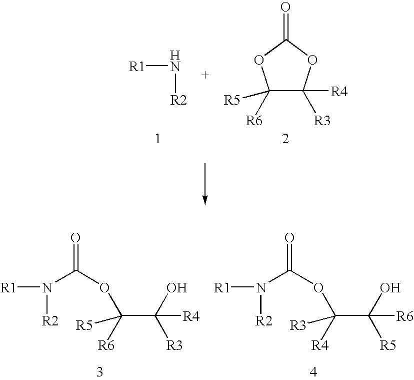

Urethane groups are useful in polymer preparation, such as polyurethanes, or other urethane-containing polymers.

Compared to ordinary urethane groups, betahydroxyurethane groups can provide desirable properties to the formed polymer, namely:
the increased resistance towards hydrolysis (Figovski, O., Improving the protective properties of non metallic corrosion resistant materials and coatings. Journal of Medeleev Chemical Society, N.Y., USA 1988 Vol 33 No 3 .pp 31-36)
more hydrophilic polymers
the extra functional groups make it possible to afterwards crosslink the polymer if desired

Another advantage of the reaction between amine and cyclic carbonate compounds is that it allows preparation of polyurethane polymers without isocyanate reactants. Such polymers are often called “non-isocyanate” polyurethane polymers. Although the use of di-isocyanates for the synthesis of polyurethanes is widely accepted throughout the industry, there are some disadvantages connected with the NCO chemistry:
di-isocyanates are toxic and need special care while handled
di-isocyanates with a low valpour pressure can be absorbed easily by the human body (skin, eyes, lungs) and are to be considered as very hazardous chemicals
their production involves phosgene while HCl is emitted: this process requires extreme safety measures
because of the hydrogenation step and the consequent purification, aliphatic di-isocyanates are expensive and often prevent the use of polyurethanes in a given application for economical reasons.

However, cyclic carbonates react relatively slowly with amines, particularly hindered primary or secondary amines and at ambient (about 25.degree. C.) temperatures. This slow reactivity has limited the usefulness of these types of reactions, for example in coatings which crosslink at ambient temperature. Therefore, means of speeding up such reactions is desired.

A great number of catalysts speeding up such reactions have been already described in literature:

U.S. Pat. No. 5,055,542 issued in 1991 recommends the use of strongly basic compounds as catalysts for reaction including less reactive secondary amines. The patent reads: “Preferred catalysts are strongly basic amines, for example diazabicyclooctane (DABCO), guanidine etc. In addition, the so-called supranucleophilic catalysts, for example 4-pyrrolidinepyridine and poly(N,N-dialkylaminopyridine) are also suitable for this purpose.” col 7, 1. 59-63).

U.S. Pat. No. 5,132,458 issued in 1992 says (col. 10, 1. 8-23): “Suitable catalysts ( . . . ) are strongly basic compounds, such as quaternary ammonium compounds, for example alkyl-, aryl- and/or benzylammonium hydroxides and carbonates. Specific representatives of quaternary ammonium compounds in this case are alkylbenzyldimethylammonium hydroxide (alkyl=C.sub.16-C.sub.22), benzyltrimethylammonium hydroxide and tetrabutylammonium hydroxide. Preferred catalysts are strongly basic amines, for example diazabicyclooctane (DABCO), guanidine, etc.

So-called supranucleophilic catalysts, for example 4-pyrrolidinopyridine and poly-(N,N-dialkylaminopyridine), are also suitable here; in this respect, cf the article by R. A. Vaidya et al. in Polymer Preprints, Vol. 2 (1986), pp. 101-102.”

On page 3 of BE 1009543 published in 1997, the classic catalysts are again mentioned: tetramethylguanidine, dimethylaminopyridine, tetrabutylammoniumhydroxide and DABCO.

U.S. Pat. No. 5,977,262, published in 1999, concerns “a process for the production of hydroxyurethanes by reacting a cyclic carbonate with a primary amine, wherein the improvement comprises, using as a catalyst a catalitically effective amount of a base whose conjugate acid has a pKa of about 11 or more.” (col. 31-36). Catalysts shown in Table 1 are: “malononitrile anion, alkyl acetoacetate anion, N-methyl ethylcarbamate anion, methoxide, hydroxide, ethoxide, t-butoxide, N-allcylamide anion, anions of benzyl alcohols, anions of alkyl or aryl ketones, diphenylamine anion, triphenylmethoxide, N,N-bis(trimethylsilyl)amide anion”. “Preferred bases are t-butoxide and N,N-bis(trimethylsilyl)amide(anion), and t-butoxide is especially preferred” (col. 2, l. 62-63). The patent further mentions that the catalyst is preferably added in the form of a salt. The salt comprises as cation acting as counterion of the base anion “an alkali metal or ammonium salt, and more preferred it is a potassium salt.”(col. 2, 1. 63-67).

However, known catalysts are either not very effective (ammoniumsalts) and/or not suited for an industrial process (very strong bases such as potassium tert-butoxide). Moreover, some reagents (amines) are not compatible with the strong bases. On the other hand, without a catalyst the reaction takes several days.

The present invention provides a process of forming an organic compound, wherein
(a) a component (A) containing at least one cyclic carbonate group having the general formula (I): 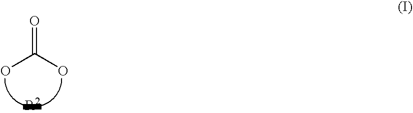
wherein:
R² represents a bivalent alkylene radical: —(CR³R⁴)ₚ— with p≧2, each R³ and each R⁴ is, independently, chosen from: hydrogen, aromatic radical, alkyl or alkenyl which contains from 0 to 8 ether bridges, and R³ and/or R⁴ may be substituted by one or more alkyl, alkenyl, aromatic radical, hydroxyl group(s), and/or cyclic carbonate group of formula (I),
(b) is reacted with a component (B) containing at least one reactive nucleophilic function X wherein each X is, independently, chosen from a primary amino or hydrazo, secondary amino or hydrazo, thiol, and/or oxime,
(c) in presence of a catalyst comprising a lithium compound
(d) to form an organic compound (C) containing at least one unit of the general formula (II): —X—CO—O—.

By component (B) containing a reactive nucleophilic function X which is a primary amino is meant any compound bearing a —NH₂ group attached directly to a carbon atom.

By component (B) containing a reactive nucleophilic function X which is a secondary amino is meant any compound bearing a —NH group attached directly to 2 carbon atoms.

By component (B) containing a reactive nucleophilic function X which is a thiol is meant any compound bearing a —SH group attached directly to a carbon atom.

By component (B) containing a reactive nucleophilic function X which is an oxime is meant any compound bearing a =N—OH group attached directly to a C atom.

The term “alkyl”, as used herein, is defined as including saturated, monovalent hydrocarbon radicals having straight, branched or cyclic moieties or combinations thereof and containing 1 to 50 carbon atoms.

The term “alkenyl” as used herein, is defined as including straight and cyclic, branched and unbranched, unsaturated hydrocarbon radicals having at least one double bond and containing from 2 to 50 carbon atoms; such as ethenyl (=vinyl), 1-methyl-1-ethenyl, 2-methyl-1-propenyl, 1-propenyl, 2-propenyl (=allyl), 1-butenyl, 2-butenyl, 3-butenyl, 4-pentenyl, 1methyl-4-pentenyl, 3-methyl-1-pentenyl, 1-hexenyl, 2-hexenyl, and the like.

The term “aryl” as used herein, is defined as including an organic radical derived from an aromatic hydrocarbon comprising 1 or more rings by removal of one hydrogen, and containing from 5 to 30 carbon atoms, such as phenyl and naphthyl.

The term “aromatic radical” as used herein, is defined as comprising a combination of alkyl or alkenyl and aryl, such as benzyl.

The term “alkylene” as used herein, is defined as including saturated, divalent hydrocarbon radicals having straight, branched or cyclic moieties or combinations thereof and containing 1 to 50 carbon atoms.

The term “alkenylene” as used herein, is defined as including unsaturated, divalent hydrocarbon radicals having straight, branched or cyclic moieties or combinations thereof, containing at least one carbon-carbon double bond and containing 1 to 50 carbon atoms.

The term “arylene” as used herein, is defined as including divalent radicals derived from an aromatic hydrocarbon comprising one or more rings by removal of two hydrogen atoms and containing from 5 to 30 carbon atoms.

The term “aralkylene” as used herein, represents a divalent radical comprising a combination of alkylene and arylene moieties.

By alkyl, alkenyl, alkylene, alkenylene, arylene and aralkylene containing an ether bridge is meant an alkyl, alkenyl, alkylene, alkenylene, arylene or aralkylene radical wherein a carbon atom is replaced by an oxygen atom, forming a group such as —C—O—C—.

By alkyl, alkylene, alkenylene, arylene and aralkylene chain containing tertiary amine bridge is meant such radical wherein a tertiary amine group is present between 2 carbon atoms, forming a group of formula —C—NR—C—, wherein R represents an alkyl or aryl group. In that case, R is preferably an alkyl group containing from 1 to 15 carbon atoms.

By alkylene, alkenylene, arylene and aralkylene containing a —CO—O— bridge is meant an alkylene, alkenylene, arylene or aralkylene radical wherein a 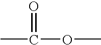
group is present between 2 carbon atoms, forming a group of formula

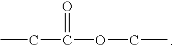

By alkylene, alkenylene, arylene and aralkylene containing a —CO— bridge is meant an alkylene, alkenylene, arylene or aralkylene radical wherein a 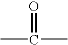
group is present between 2 carbon atoms.

Advantages obtainable by the claimed process include:
An increase of reactionspeed during the synthesis of (poly)urethanes by reacting (poly)amines with (poly)cyclocarbonates (compared to prior art catalysts and to non-catalized reactions)
Compatibility with many commercial amines such as JEFFAMINES (polyoxyalkyleneamines available from Huntsman Corp), in contrast with the preferred catalyst of the prior art patent U.S. Pat. No. 5,977,262, which is a strong base: Potassium tertiobutoxy (also called K t-butoxide).
cheap, commercially available (poly)amines can be used for the synthesis of (poly)urethanes. These amines include JEFFAMINE, diethanolamine, piperazine, . . . .
Because the reactionspeed is increased considerably even hindered primary and secondary amines can be used in the synthesis.
Shorter reactiontimes also lead to an economical advantage.
The new catalyst is a non-volatile and relatively safe reagent. For example lithium bromide has been used as a tranquilizer and lithium carbonate and lithium benzoate have also been used as drugs.

In component (A), as specified above, each R³ and each R⁴ are chosen independently from each other. This means that each R³ is chosen independently from other R³ and from R⁴ and that each R⁴ is chosen independently from other R⁴ and from R³ on the same carbon as well as on different carbon atoms of the cycle.

The component (A) can comprise at least one carbonate cycle having 5, 6 or even more members in his ring. Preferably, the component (A) contains at least one 5-membered cyclic carbonate group. This means that p=2 in general formula (I). Such 5-membered cyclic carbonate compounds are very desirable because they can be easily prepared starting from cheap epoxides in a reaction with carbondioxide. A reaction of which is already known that it can be catalysed by a lithiumsalt: ref Kihara, N., Hara, N., Endo, T.; J. Org Chem., 1993, 58, 6198-6202.

When component (A) comprises only one carbonate cycle, p is preferably 2 or 3, most preferably 2. Particularly preferred cyclic carbonates of this type are those wherein one of the R³ substituents is chosen from the group of hydrogen, methyl, ethyl, hydroxymethyl, chloromethyl, allyloxymethyl, and wherein the R⁴ substituent present on the same carbon atom as this R³ substituent is chosen from hydrogen and methyl, all other R³ and all other R⁴ substituents being hydrogen. For example component (A) can be: propylene carbonate, ethylene carbonate (formulas shown in the examples) and butylenecarbonate(4-ethyl-1,3-dioxolan-2-one), glycerinecarbonate(4-hydroxymethyl-1,3-dioxolan-2-one), allyloxymethylcarbonate(4-allyloxymethyl-1,3-dioxolan-2-one), epichlorohydrin carbonate (4-chloromethyl-1,3-dioxolan-2-one) and neopentylglycol carbonate(5,5-dimethyl-1,3-dioxan-2-one).

More preferably, the component (A) contains at least two carbonate cycles. This permits to obtain dimers, oligomers or polymers, after rings openings. Components (A) containing at least two carbonate cycles are preferably chosen from those of formula (IV), (V) and (VI) 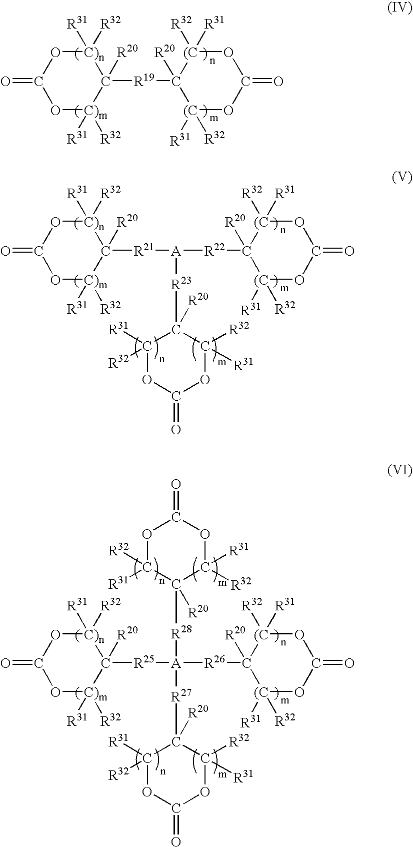
wherein n=0 to 2, m=0 to 2, n+m≧1, and
each R³¹, each R³², each R²⁰ is, independently, chosen from the group of
   hydrogen,
   halogen,
   hydroxy,
   alkyl, optionally substituted by hydroxy; halogen; aryl and/or aryl substituted by hydroxy, halogen or alkyl; and optionally containing from 1 to 8 ether bridges,
   alkenyl, optionally substituted by hydroxy; halogen; aryl and/or aryl substituted by hydroxy, halogen or alkyl; and optionally containing from 1 to 8 ether bridges,
   aryl, optionally substituted by hydroxy; halogen; alkyl; alkyl substituted by hydroxy, halogen and/or aryl; and/or alkyl containing from 1 to 8 ether bridges,
R¹⁹, R²¹, R²², R²³, R²⁵, R²⁶, R²⁷ and R²⁸, are, independently, chosen from alkylene, alkenylene, arylene and aralkylene chains which may contain from 1 to 20 ether bridges, from 1 to 4 tertiary amine bridges, from 1 to 4 —CO— bridges and/or from 1 to 4 —O—CO— bridges; 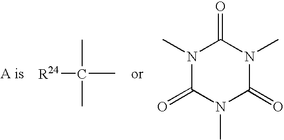
wherein R²⁴ is hydrogen or alkyl;

In the cyclic carbonates of formula (IV), (V) and (VI) n and m are, independently, preferably 0 or 1. In the cyclic carbonates of formula (IV), (V) and (VI) n+m is preferably 1.

In the cyclic carbonates of formula (IV), (V) and (VI) each R³¹ and each R³² is, independently, preferably chosen from the group of hydrogen; alkyl comprising from 1 to 6 carbon atoms, optionally substituted by hydroxy or halogen; and alkenyl comprising from 1 to 6 carbon atoms; both optionally containing from 1 to 3 ether bridges. Most preferably, all but one of the R³¹ substituents are hydrogen. In these preferred cyclic carbonates, all but one of the R³² substituents are preferably hydrogen. Most preferably, all R³² substituents are hydrogen. Particularly preferred cyclic carbonates of formula (IV), (V) and (VI) are those wherein each R³¹ and each R³² is hydrogen.

In the cyclic carbonates of formula (IV), (V) and (VI) each R²⁰ is preferably hydrogen.

In the cyclic carbonates of formula (IV) R¹⁹ is preferably chosen from alkylene and aralkylene chains which may contain from 1 to 20 ether bridges. Preferred are alkylene and aralkylene chains comprising at least 2 ether bridges.

In the cyclic carbonates of formula m and (VI) R²¹, R²², R²³, R²⁵, R²⁶, R²⁷and R²⁸, are preferably, independently, chosen from alkylene and aralkylene chains which may contain from 1 to 3 ether bridges.

In the cyclic carbonates of formula (V) used in the process according to the invention R²⁴ is preferably hydrogen or alkyl comprising form 1 to 4 carbon atoms.

Particularly preferred components (A) are chosen from propylene carbonate, ethylene carbonate, butylenecarbonate, glycerinecarbonate, allyloxymethylcarbonate and biscarbonates made starting from the diglycidylethers of bisphenol A 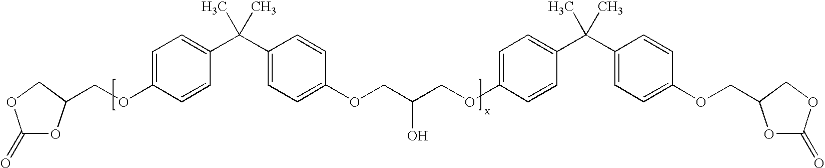
wherein x≧0, preferably x=0 to 15 and most preferably x=0; or made starting from the diglycidyl ether of polypropylene glycol 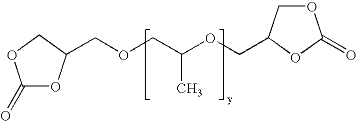
wherein y≧2, preferably y=3 to 40, most preferably y=3 to 20.

Component (A) can be easily prepared by bubbling carbon dioxide through (poly-)epoxy compounds in the presence of a catalyst. Examples of useful (poly)epoxide compounds' include, without limitation, polyglycidyl ethers and esters, epoxy novolac resins, and epoxide-functional acrylics. In particular, the polyepoxide compound may be the polyglycidyl ether of aliphatic or aromatic polyols such as 1,4-butanediol, neopentyl glycol, cyclohexane dimethanol, diethylene glycol, polyethylene glycol, dipropylene glycol, polypropylene glycol, 2,2,4-trimethyl-1,3-pentanediol, 1,6-hexanediol, trimethylolpropane, trimethylolethane, glycerol, bisphenol A(4,4′-isopropylidenediphenol), 1,1,1-tris(4-hydroxyphenyl)ethane, hydroquinone, 4,4′-biphenol, 2,2′-biphenol, 4,4′-dihydroxybenzophenone, 1,5-dihydroxynaphthylene, novolac polyphenols, resorcinol, and similar compounds. In principle, the glycidyl ether of any polyol can be used.

The polyepoxide compound is preferably a polyglycidyl ether of a polyphenol, and particularly preferably, it is the diglycidyl ether of bisphenol A. The polyepoxide compound could also be extended, for example by reaction of the diglycidyl ether of bisphenol A with a polyphenol such as bisphenol or with a polyamine such as those sold by BASF AG of Germany under the tradename POLYAMIN and under the tradename Jeffamine.RTM. by Huntsman Co. of Houston, Tex.

Preferably, the component (B) contains at least one nucleophilic function X which is an amino group.

Preferably, component (B) is an amine of formula (IX), (X), (XI) or (XII) 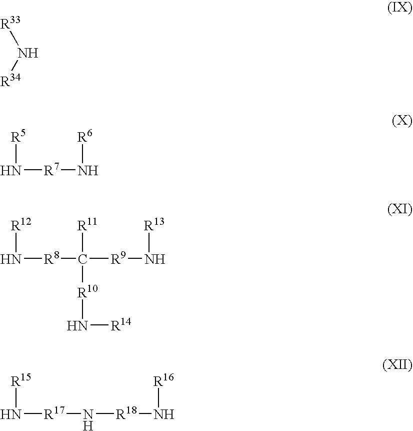
wherein
R³³ is an alkyl, optionally substituted by hydroxy, tertiary amine and/or aryl, and optionally containing from 1 to 20 ether bridges and/or from 1 to 3 tertiary amine bridges, R³⁴, R⁵, R⁶, R¹², R¹³, R¹⁴, R¹⁵ and R¹⁶ are, independently, chosen from the group of
   hydrogen, and
   alkyl, optionally substituted by hydroxy, tertiary amine and/or aryl, and optionally containing from 1 to 8 ether bridges and/or from 1 to 3 tertiary amine bridges,
   with the proviso that, respectively, R³³ and R³⁴, R⁵ and R⁶, R¹² and/or R¹³ and/or R¹⁴, R¹⁵ and R¹⁶ may be linked together in order to form a ring,
R⁷, R⁸, R⁹, R¹⁰, R¹⁷ and R¹⁸ are, independently, chosen from alkylene, alkenylene, arylene and aralkylene chains which may contain from 1 to 8 ether bridges and/or from 1 to 3 tertiary amine bridges,
R¹¹ is hydrogen or alkyl.

In the amines of formula (IX) R³³ is preferably an alkyl, optionally substituted by hydroxy, tertiary amine and/or aryl, and optionally containing from 1 to 20 ether bridges. Most preferably, R³³ is chosen from the group of alkyl comprising up to 10 carbon atoms, optionally substituted by one hydroxy or tertiary amine and/or optionally containing one or two ether bridges. Non-limiting examples are R³³ substituents chosen from the group of n-propyl, isopropyl, n-butyl, isobutyl, sec-butyl, tert-butyl, n-hexyl, n-octyl, 2-ethylhexyl, isononyl, cyclopentyl, cyclohexyl, 2-methylcyclohexyl, N,N-(di-tert-butyl)ethyl, benzyl, 2-(2-hydroxyethoxy)ethyl, 5-hydroxypentyl, 2-hydroxyethyl, 2-hydroxypropyl, 3-hydroxypropyl, 3-(diethylamino)propyl, 2-(diethylamino)ethyl, 1-methyl-4-(diethylamino)butyl, 2-((di-tert-butyl)amino)ethyl, 3-(dimethylamino)propyl, 2-methoxyethyl, 2-ethoxyethyl, 3-methoxypropyl, 2-methoxyisopropyl, 3-ethoxypropyl, 3-isopropoxypropyl, 3-(2-methoxyethoxy)propyl, 3-(2-ethylhexyloxy)propyl, CH₃O(CH₂CH₂O)₆—(CH₂CHR—O)₁₀—CH₂—CH(CH₃)— wherein R is H or CH₃ in a proportion of 1:9, ethyl, methyl, 1,2-dimethylpropyl.

In the amines of formula (IX) R³⁴ is preferably chosen from the group of hydrogen and alkyl, optionally substituted by hydroxy, tertiary amine or aryl, and optionally containing from 1 to 8 ether bridges. Most preferably, R³⁴ is chosen from the group of hydrogen and alkyl comprising up to 10 carbon atoms, optionally substituted by one hydroxy or tertiary amine and/or optionally containing one or two ether bridges. Non-limiting examples are R³⁴ substituents chosen from the group of hydrogen, ethyl, n-propyl, isopropyl, n-hexyl, methyl, tert-butyl, n-butyl, isobutyl, n-octyl, 2-ethylhexyl, 1,2-dimethylpropyl, cyclohexyl, 2-hydroxyethyl, 2-hydroxyisopropyl, 3-hydroxypropyl, 2-methoxyethyl, 3-(dimethylamino)propyl.

In the amines of formula (X), (XI) and (XII) R⁵, R⁶, R¹², R¹³, R¹⁴, R¹⁵ and R¹⁶ are preferably, independently, chosen from the group of hydrogen and alkyl comprising up to 10 carbon atoms, most preferably up to 6 carbon atoms.

In the amines of formula (IX), (X), (XI) and (XII) R³³ and R³⁴, R⁵ and R⁶, R¹² and/or R¹³ and/or R¹⁴, R¹⁵ and R¹⁶, respectively, may be linked together in order to form a ring. In this case, these substituents are preferably linked so that they form an alkylene chain comprising from 2 to 7 carbon atoms, and optionally containing 1 or 2 ether bridges. In case of R³³ and R³⁴, this alkylene chain is preferably such that a 5 to 7-membered ring is formed, for example a pyrolidine ring, a piperidine ring or a morpholine ring, which may further be substituted by alkyl groups. In case of R⁵ and R⁶, this alkylene chain is preferably such that a 5 to 7-membered ring is formed, for example piperazine, which may further be substituted by alkyl groups.

In the amines of formula (X) R⁷ is preferably chosen from the group of alkylene and aralkylene chains, containing up to 20 carbon atoms and which may contain from 1 to 8 ether bridges and/or from 1 to 3 tertiary amine bridges. Most preferably, R⁷ is chosen from the group of ethylene, 1,2-propylene, trimethylene, hexamethylene, 2,2-dimethylpropylene, 1-methyltrimethylene, 1,2,3-trimethyltetramethylene, 2-methyl-pentamethylene, 2,2,4-(or 2,4,4-)trimethylhexamethylene, metaxylylene, 3, 5,5-trimethylcyclohexyl-1-ene-3-methylene, bis(cyclohexyl-4-ene)methane, bis(4-methylcyclohexyl-3-ene)methane, cyclohexyl-1,3-ene, cyclohexyl-1,4-ene, 1,4-bis(propoxyl-3-ene)butane, N,N-bis(trimethylene)methylamine, 3,6-dioxaoctylene, 3,8-dioxadodecylene, 4,7,10-trioxatridecylene, poly(oxytetramethylene), poly(oxypropylene) with 2 to 15 1,2-propylene oxide units, poly(oxypropylene-co-oxyethylene) with 2 to 15 propylene oxide and 2 to 15 ethylene oxide units, 2,2-dimethylpropylene.

In the amines of formula (XI) R⁸, R⁹, R¹⁰ are preferably, independently, chosen from the group of alkylene, optionally containing from 1 to 8 ether bridges. Most preferably R⁸, R⁹, R¹⁰ are chosen from alkylene comprising up to 15 carbon atoms and containing up to 5 ether bridges.

In the amines of formula (XII) R¹⁷ and R¹⁸ are preferably, independently, chosen from the group of alkylene. Most preferably R¹⁷and R¹⁸ are chosen from alkylene comprising up to 6 carbon atoms.

In the amines of formula (XI) R¹¹ is preferably hydrogen or an alkyl containing from 1 to 4 carbon atoms.

Amines of formula (IX), (X), (XI) and (XII) are known in the art. Amines of formula (IX) which are particularly useful in the process according to the invention are n-propylamine, isopropylamine, n-butylamine, isobutylamine, sec-butylamine, tert-butylamine, 3-methylbutylamine, n-hexylamine, n-octylamine, 2-ethylhexylamine, isononylamine, cyclopentylamine, cyclohexylamine, 2-methylcyclohexylamine, N,N-(di-tert-butyl)ethyleneamine, benzylamine, 2-(2-aminoethoxy)ethanol, 5-aminopentanol, ethanolamine, 1-aminopropan-2-ol, 3-amino-1-propanol, 3-(diethylamino)propylamine, 2-(diethylamino)ethylamine, 1-methyl-4-(diethylamino)butylamine, 2,2-(di-tert-butylamino)ethylamine, 3-(dimethylamino)propylamine, 2-methoxyethylamine, 2-ethoxyethylamine, 3-methoxypropylamine, 1-methoxyisopropylamine, 3-ethoxypropylamine, 3-isopropoxypropylamine, 3-(2-methoxyethoxy)propylamine, 3-(2-ethylhexyloxy)propylamine, polyoxyalkylene amines such as α-oxymethyl-ω-(2-propylamino)-poly(oxypropylene-co-oxyethylene) with an average number of 1,2-propylene oxide units of 9 and an average number of ethylene oxide units of 7, also known as Jeffamine® M-600 (manufactured by Hunstman), diethylamine, di-n-propylamine, diisopropylamine, di-n-hexylamine, N-methylbutylamine, N-ethylbutylamine, di-n-butylamine, diisobutylamine, di-n-octylamine, bis(2-ethylhexyl)amine, N-ethyl-1,2-dimethylpropylamine, dicyclohexylamine, cyclohexylmethylamine, cyclohexylethylamine, N-methylbenzylamine, 2-methylaminoethanol, 2-ethylaminoethanol, 2-butylaminoethanol, diethanolamine, diisopropanolamine, 3-(2-hydroxyethyl)aminopropanol, bis(2-methoxyethyl)amine, bis(3-dimethylaminopropyl)amine, pyrolidine, piperidine, morpholine, 2,6-dimethylmorpholine.

Amines of formula (X) which are particularly useful in the process according to the invention are ethylenediamine, 1,2-propylenediamine, trimethylenediamine, hexamethylenediamine, 2,2-dimethylpropane-1,3-diamine, 1-methyl-1,3-propanediamine, 1,2,3-trimethyl-1,4-butanediamine, 2-methyl-1,5diaminopentane, 2,2,4-(or 2,4,4-)trimethylhexamethylene diamine, metaxylylenediamine, 1-amino-3-aminomethyl-3,5,5trimethylcyclohexane(isophorone diamine), bis-(4-aminocyclohexyl)-methane, bis-(4-amino-3-methyl-cyclohexyl)-methane, 1,3-cyclohexanediamine, 1,4-cyclohexanediamine, 1,4-Bis(3-aminopropoxy)butane diamine, N,N-bis(3-aminopropyl)methylamine, triethyleneglycol diamine, 3,3′-(butane-1,4-diylbis(oxy))bispropaneamine, 4,7,10-trioxatridecan-1,13-diamine, polyoxyalkylene diamines such as α-amino-ω-(4-butylamino)-poly(oxytetramethylene), α-amino-ω-(2-propylamino)-poly(oxypropylene) with an average number of 1,2-propylene oxide units of 2.6, also known as Jeffamine® D-230 (manufactured by Hunstman), α-amino-ω-(2-propylamino)-poly(oxypropylene) with an average number of 1,2-propylene oxide units of 5.6, also known as Jeffamine® D-400 (manufactured by Hunstman), α-amino-ω-(2-propylamino)-poly(oxypropylene-co-oxyethylene) with an average number of 1,2 propylene oxide units of 2.5 and ethylene oxide units of 8.5, also known as Jeffamine® ED-600 (manufactured by Hunstman), 2,2-dimethyl-1,3-propanediamine, N,N′-di-tert-butyl-ethanediamine, N,N′-dimethylhexyl-1,6-diamine, piperazine, 1,6-diamino-trimethylhexane, N,N′-dimethyl-1,3-propanediamine and 2,5-dimethylpiperazine.

Amines of formula (XI) that are particularly useful in the process according to the invention is propoxylated trimethylopropane triamine with an average number of number of 1,2-propylene oxide units of 5.3, also known as Jeffamine® T-403 (manufactured by Hunstman).

Amines of formula (XII) that are particularly useful in the process according to the invention are diethylenetriamine, N,N-dimethyldipropylenetriamine, bis(hexamethylene)triamine.

In an embodiment of the invention, compound (B) contains only one primary or secondary amino group.

In other preferred embodiments of the invention, component (B) is a compound containing at least two primary or secondary amino groups. This permits to obtain dimers, oligomers and even polymers when reacted with a component (A) containing at least two linked carbonate rings.

Especially preferred are amines chosen amongst cyclohexylamine, N-methylbutylamine, N-methylbenzylamine, piperidine, piperazine, morpholine, benzylamine, diethylenetriamine, ethanolamine, diethanolamine and polyoxyalkylene amines and diamines.

The component (B) can also be chosen among hydrazo compounds such as: hydrazine, mono, di and tri-substituted hydrazines H₂N—NHR²⁹, H₂N—NR²⁹R³⁰, R²⁹HN—NHR³⁰, R²⁹HN—NR³⁰R³⁵ wherein R²⁹, R³⁰ and R³⁵ are as defined for R³³.

The lithium compound used as catalyst in the present invention is especially in form of a salt LiₘA comprising lithium as the cation (mLi⁺) and an anion as counterion (A^{m-}). It is preferred that the lithium compound is lithium oxide (Li₂O), lithium hydroxide (LiOH), lithium carbonate (Li₂CO₃), methoxylithium (LiOCH₃), tertiobutoxylithium (LiOtBu), lithium citrate, lithium chloride (LiCl), Li-stearate (LiC₁₈H₃₅O₂), LiClO₄, Li₂SO₄, LiOAc, LiOOCPh and/or lithium bromide (LiBr). Especially preferred ones are Li₂O and LiBr. LiBr, Li₂CO₃, Li citrate are advantageous because of their non-toxicity.

It was found that the reaction temperature is not critical and can be comprised between 0 and 120° C., preferably between 50 and 80° C.

The amount of component (A) and component (B) in the process according to the invention is preferably such that the molar ratio of cyclic carbonate groups to nucleophilic groups X is from 0.5 to 2. In case that a component (A) comprising only one cyclic carbonate group and/or a component (B) comprising only one nucleophilic group X is used, the ratio of amount of such components is preferably such that an almost equivalent ratio of cyclic carbonate groups to nucleophilic groups X is obtained.

Preferably the catalyst concentration is comprised between 0.01 and 5% by weight of the reacting mixture, preferably between 0.1 and 2% by weight of the reacting mixture. Such concentration was found sufficient for an effective speeding up of the reaction.

The reaction can be made with or without solvent. When a solvent is used, it is preferably chosen among alcohol, ether, ester, dimethylformamide and water.

Furthermore, component (A) containing at least one cyclic carbonate compound can be prepared by reaction of the corresponding epoxide compound with carbon dioxide (CO2) in presence of a lithium compound as catalyst. This is particularly advantageous in industrial practice because the same catalyst can be used for successive reactions (“one-pot synthesis”).

The invention also extends to a polymeric composition containing a nonisocyanate polyurethane polymer comprising urethane functions —NCOO— which contain ternary-substituted nitrogen and bear an hydroxy group in beta-position (betahydroxyurethane).

The invention further extends to the use of a lithium compound to catalyse a ring opening reaction wherein:
a component (A) containing at least one cyclic carbonate group having the general formula (I): 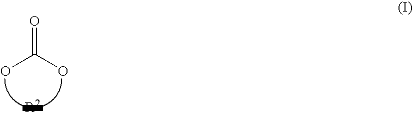
wherein:
R² represents a bivalent alkylene radical: —(CR³R⁴)ₚ— with p≧2,
each R³ and each R⁴ is independently chosen from: hydrogen, aromatic radical, alkyl or alkenyl which contains from 0 to 8 ether bridges, and R³ and/or R⁴ may be substituted by one or more alkyl, alkenyl, aromatic radical, hydroxyl group(s), and/or cyclic carbonate group of formula (I), is reacted with a component (B) containing at least one reactive nucleophilic function X wherein each X is, independently, chosen from a primary amino or hydrazo, secondary amino or hydrazo, thiol, and/or oxime,
to form an organic compound (C) containing at least one unit of the general formula (II):
   —C—CO—O—.

The invention will now be illustrated by examples.

Examples 1 to 6 shows the reaction of diethanolamine and propylenecarbonate, without catalyst, with prior art catalyst and with catalyst according to the invention. Example 7 to 10 shows the reaction of Jeffamine M600 with propylenecarbonate. Example 11 is a comparative example showing one disadvantage of a prior art catalyst. Example 12 shows the preparation of a tricarbamate by reaction of diethylenetriamine with ethylenecarbonate.

### EXAMPLE 1-6

### Reaction of Diethanolamine and Propylenecarbonate

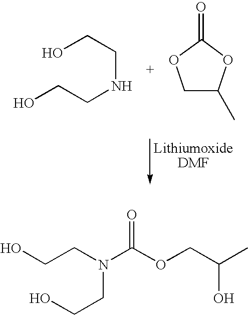

To a suitable 3-neck flask equipped with a stirrer and a thermometer were added 105 grams (1 mole) of diethanolamine, 1200 ml of dimethylformamide and 204 grams (2 moles) of propylenecarbonate. The reaction was run at roomtemperature and followed by potentiometric titration of the amine. All examples were run in the same manner except for varying and/or amount of catalyst present. Results for the examples are shown in table 1.

TABLE 1

| | | Time (min)^{b} | | | | |
|---|---|---|---|---|---|---|
| Example | Catalyst^{a} | 0 | 5 | 10 | 15 | 20 |
| 1 | none | 1.00 | 0.99 | 0.98 | 0.97 | 0.97 |
| 2 | 0.2% BtMACl | 1.00 | 0.98 | 0.97 | 0.96 | 0.96 |
| 3 | 2% BtMACl | 1.00 | 0.98 | 0.96 | 0.95 | 0.94 |
| 4 | 0.2% BtMAOH | 1.00 | 0.88 | 0.84 | 0.81 | 0.80 |
| 5 | 0.2% K | 1.00 | 0.63 | 0.37 | 0.25 | 0.22 |
| | tertiobutoxy | | | | | |
| 6 | 0.2% Li₂O | 1.00 | 0.56 | 0.30 | 0.18 | 0.16 |

^{b}numbers given are relative (to time zero) amounts of amine remaining at the given time
^{a}on total mass of sample
BtMACl = benzyltrimethylammonium chloride-
BtMAOH = benzyltrimethylammonium hydroxide

### EXAMPLE 7-10

### Reaction of JEFFAMINE M600 with Propylenecarbonate

Benzyltrimethylammoniumchloride=BtMACl.

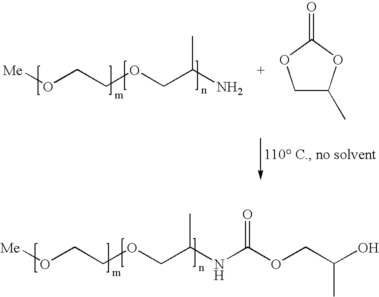

To a solution of 20 grams of JEFFAMINE RTM (Registered TradeMark) M600, (polyetheramine from Texaco, having an EO/PO ratio of 10/90 and molecular weight of 600 e from Huntsman Corp., Salt Lake City, Utah) was added 6,8 grams of propylenecarbonate and a catalyst if appropriate. The reaction was run at 110° C. and followed by potentiometric titration of the amine. All examples were run in the same manner except for varying and/or amount of catalyst present. Results for the examples are shown in table 2.

TABLE 2

| | | Time (min)^{a} | | | | |
|---|---|---|---|---|---|---|
| Example | Catalyst | 0 | 30 | 60 | 90 | 120 |
| 7 | none | 1.00 | 0.90 | 0.83 | 0.81 | 0.79 |
| 8 | BtMACl^{b} | 1.00 | 0.87 | 0.79 | 0.72 | 0.68 |
| 9 | BtMAOH^{b} | 1.00 | 0.81 | 0.70 | 0.60 | 0.53 |
| 10 | LiBr^{b} | 1.00 | 0.73 | 0.56 | 0.45 | 0.39 |

^{a}numbers given are relative (to time zero) amounts of amine remaining at the given time
^{b}3.5% on total weight

### EXAMPLE 11

### Comparative Example with Catalyst from U.S. Pat. No. 5,977,262

To a solution of 20 grams of JEFFAMINE RTM M600, (polyetheramine from Texaco, having an EO/PO ratio of 90/10 and molecular weight of 600 sold by Huntsman Corp., Salt Lake City, Utah), 3.4 grams of propylenecarbonate and 100 mg IRGANOX 1010 RTM of Ciba, Basel, Switzerland) is added 500 mg potassium tert-butoxide as a catalyst.

After some time the reaction mixture develops the strong and characteristic smell of amonia and shows discoloration (turns dark brown), indicating degradation of the amine component and formation of side-product(s).

### EXAMPLE 12

### Reaction of Diethylene Triamine with Ethylenecarbonate

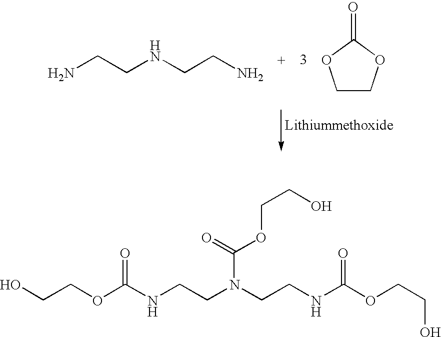

To a suitable 3-neck flask equipped with a stirrer and a thermometer were added 103 grams (1 mole) of diethylenetriamine and 264 grams (3 moles) of ethylene carbonate. At the completion of the ethylene carbonate addition the reaction mixture was allowed to react for several hours at 65° C. The yellow product was characterized by I.R. and potentiometric titration to be mainly tris(2-hydroxyethyl)iminodiethylene triscarbamate.

In the absence of a catalyst, the secundary amine diethylenetriamine is unreactive towards cyclic carbonates.

1. Process of forming an organic compound, wherein
(a) a component (A) containing at least one cyclic carbonate group having the general formula (I): 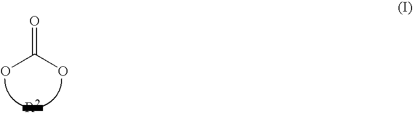
wherein:
R² represents a bivalent alkylene radical: —(CR³R⁴)ₚ— with p≧2,
each R³ and each R⁴ is, independently, chosen from: hydrogen, aromatic radical, alkyl or alkenyl which contains from 0 to 8 ether bridges, and R³ and/or R⁴ may be substituted by one or more alkyl, alkenyl, aromatic radical, hydroxyl group(s), and/or cyclic carbonate group of formula (I),
(b) is reacted with a component (b) containing at least one reactive nucleophilic function X wherein each X is, independently, chosen from a primary amino or hydrazo, secondary amino or hydrazo, thiol and/or oxime,
(c) in presence of a catalyst comprising a lithium compound
(d) to form an organic compound (c) containing at least one unit of the general formula (II): —X—CO—O—.

2. Process according to claim 1, wherein component (A) contains at least one 5-membered cyclic carbonate group (p=2 in general formula (I)).

3. Process according to claim 1, wherein component (A) contains at least two carbonate cycles.

4. Process according to claim 1, wherein component (A) is chosen from propylene carbonate, ethylene carbonate, butylenecarbonate, glycerinecarbonate, allyloxymethylcarbonate and biscarbonates made starting from the diglycidylethers of bisphenol A or of polypropylene glycol.

5. Process according to claim 1, wherein component (B) contains at least one nucleophilic function X which is an amino group.

6. Process according to claim 5, wherein component (B) is an amine of formula (IX), (X), (XI) or (XII) 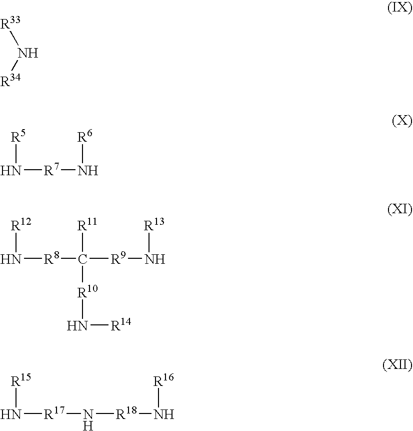
wherein
R³³ is an alkyl, optionally substituted by hydroxy, tertiary amine and/or aryl, and optionally containing from 1 to 20 ether bridges and/or from 1 to 3 tertiary amine bridges,
R³⁴, R⁵, R⁶, R¹², R¹³, R¹⁴, R¹⁵ and R¹⁶ are, independently, chosen from the group of
hydrogen, and
alkyl, optionally substituted by hydroxy, tertiary amine and/or aryl, and optionally containing from 1 to 8 ether bridges and/or from 1 to 3 tertiary amine bridges,
with the proviso that, respectively, R³³ and R³⁴, R⁵ and R⁶, R¹² and/or R¹³ and/or R¹⁴, R¹⁵ and R¹⁶ may be linked together in order to form a ring,
R⁷, R⁸, R⁹, R¹⁰, R¹⁷ and R¹⁸ are, independently, chosen from alkylene, alkenylene, arylene and aralkylene chains which may contain from 1 to 8 ether bridges and/or from 1 to 3 tertiary amine bridges,
R¹¹ is hydrogen or alkyl.

7. Process according to claim 5, wherein component (B) contains at least two primary or secondary amino groups.

8. Process according to claim 6, wherein component (B) is an amine chosen amongst cyclohexylamine, N-methylbutylamine, N-methylbenzylamine, piperidine, piperazine, morpholine, benzylamine, diethylenetriamine, ethanolamine, diethanolamine and polyoxyalkylene amines and diamines.

9. Process according to claim 1, wherein the lithium compound is lithium oxide (Li₂O), lithium hydroxide (LiOH), lithium carbonate (Li₂CO₃), methoxylithium (LiOCH₃), terbutoxylithium (LiOtBu), lithium citrate, lithium chloride (LiCl), Li-stearate (LiC₁₈H₃₅O₂), LiClO₄, Li₂SO₄, LiOAc, LiOOCPh and/or lithium bromide (LiBr).

10. Process according to claim 1, wherein the reaction temperature is comprised between 0 and 120° C.

11. Process according to claim 1, wherein the amount of component (A) and component (B) are such that the molar ratio of cyclic carbonate groups to nucleophilic groups X is from 0.5 to 2.

12. Process according to claim 1, wherein the catalyst concentration is comprised between 0.01 and 5% by weight of the reacting mixture.

13. Process according to claim 12, wherein the catalyst concentration is comprised between 0.1 and 2% by weight of the reacting mixture.

14. Process according to claim 1, wherein the reaction is made in a solvent chosen among: alcohol, ether, ester, dimethylformamide, and water.

15. Process according to claim 1, wherein component (A) containing at least one cyclic carbonate compound is prepared by reaction of the corresponding epoxide compound with carbon dioxide (CO₂) in presence of a lithium compound as catalyst.

16. A method of conducting a ring opening reaction wherein:
a component (A) containing at least one cyclic carbonate group having the general formula (I): 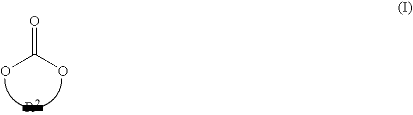
wherein:
R² represents a bivalent alkylene radical: —(CR³R⁴)ₚ— with p≧2,
each R³ and each R⁴ is independently chosen from: hydrogen, aromatic radical, alkyl, alkenyl which contains from 0 to 8 ether bridges, and R³ and/or R⁴ may be substituted by one or more alkyl, alkenyl, aromatic radical, hydroxyl group(s), and/or cyclic carbonate group of formula (I),
is reacted with a component (B) containing at least one reactive nucleophilic function X wherein each X is, independently, chosen from a primary amino or hydrazo, secondary amino or hydrazo, thiol, and/or oxime, in the presence of a lithium compound as catalyst
to form an organic compound (C) containing at least one unit of the general formula (II): —X—CO—O—.

17. Process according to claim 10 wherein the reaction temperature is 50 to 80° C.
